(19) United States
(12) Patent Application Publication
(10) Pub. No.: US 2012/0275998 A1
(43) Pub. Date: Nov. 1, 2012

(54) BIODEGRADABLE STENT COMPRISING AN ACID SCAVENGING AGENT

(76) Inventor: Kadem Gayad Al-Lamee, Leeds (GB)

(21) Appl. No.: 13/512,741

(22) PCT Filed: Dec. 2, 2010
(86) PCT No.: PCT/GB10/02219

§ 371 Date: Jul. 18, 2012

(30) Foreign Application Priority Data
Dec. 3, 2009 (GB) 0921203.6

(51) Int. Cl. A61K 31/519; A61K 47/30; A61K 49/18; A61K 31/522; A61K 51/00

(52) U.S. Cl. 424/1.11; 514/262.1; 514/263.34; 424/9.3; 514/772.3

(57) ABSTRACT

A biodegradable stent comprising a biodegradable material having dissolved therein an acid scavenging agent. The biodegradable material may be PLLA or PLGA. The acid scavenging agent may be also a pharmaceutical agent, for example an antiproliferative agent, coronary vasodilator agent and/or a bronchodilator. Preferably the acid scavenging agent is dipyridamole and/or mopidamol. The invention also provides a method of preparing a biodegradable material for use in the stent of the invention comprising: (i) preparing a formulation of the biodegradable material and the acid scavenging agent; (ii) heating the formulation to melt the biodegradable material and the acid scavenging agent so as to dissolve the agent in the material; and (iii) collecting and cooling the formulation of step (ii).

A leading cause of mortality within the developed world is cardiovascular disease. Coronary disease is of most concern. Patients having such disease usually have narrowing in one or more coronary arteries. One treatment is coronary stenting, which involves the placement of a stent at the site of acute artery closure. This type of procedure has proved effective in restoring vessel patency and decreasing myocardial ischemia.

However the exposure of currently used metallic stents to flowing blood can result in thrombus formation, smooth muscle cell proliferation and acute thrombotic occlusion of the stent.

In view of the problems associated with using exposed metallic stents, much effort has been made to develop stents having a biocompatible coating, a drug-eluting stent and fully degradable stents. Many such stents are commercially available and one of the full biodegradable stent is currently undergoing clinical trials (for example, the Abbott® ABSORB trial of a stent made of a biodegradable polyester derived from lactic acid with a coating that controls release of the drug everolimus to prevent rejection and reclogging).

However a problem with existing biodegradable stents is that the degradation products of the biodegradable material use can cause an inflammatory response of tissue adjacent to the site of the stent.

A solution has been proposed to this problem using anti-inflammatory agents coated on the outside of the stent. However, it can be appreciated that this approach simply treats the symptoms of the inflammatory response. It would be advantageous to prevent the degradation products of the biodegradable material inducing an inflammatory response, rather than simply alleviating the effect of the degradation products.

The inventor has also investigated the means by which pharmaceutically active agents are delivered with a stent.

It is known in the art for stents to be used to deliver pharmaceutically active agents to a patient. Such stents contain a reservoir of pharmaceutically active agents, the agent being released over time according to the means by which the agent is formulated with the stent.

It is known to prepare stents in which such pharmaceutically active agents are coated on to a surface of the stent. Optionally different amounts of the agent can be coated on to different surfaces of the stent. For example, the outside surface of the stent may have more agent than the interior of the stent.

Pharmaceutically active agents may also be included at locations within the stent. For example, some stents are prepared comprising a mixture of different materials, each of which can have pharmaceutically active agents coated thereon.

Some stents are prepared to provide a matrix or voids in the structure of the stent, and pharmaceutically active agents can be placed within that matrix or void.

Still further stents are prepared in which the pharmaceutically active agent is provided in microcapsules embedded within the matrix or voids.

However the present inventor has realised that there are disadvantages associated with each of these methods. In particular, existing methods of associating pharmaceutically active agents with stents require the agents to be mixed with polymers and dissolved in organic solvents and then applied in the coating process of the stents, which require a further stage in the manufacturing process of the stent. The coating can be delaminated and can have adverse biological effects. Moreover, differential loading of pharmaceutically active agent in stents can lead to undesirable unpredictability for the elution of the pharmaceutically active agent from the stent.

Against this background there is a continuing need to devise stents which are both more biologically acceptable to the implanted patient and have improved pharmaceutically active agent release characteristics.

A first aspect of the invention provides a biodegradable stent comprising a biodegradable material having dissolved therein an acid scavenging agent.

The stent of the invention provides the following advantages over stents known in the art.

It is known that the in situ degradation of polymers used in biodegradable stents by water hydrolysis can cause local inflammation. This is due to the acidity of the breakdown products. As mentioned above, it has previously been proposed to address this problem by incorporating anti-inflammatory agents within the stent. However as can be appreciated such an approach merely treats the symptoms of the response of the body to the acidic breakdown problems. Moreover this approach can require the use of biological medicines or other agents which can add costs and complications to the process of manufacturing the stent.

The present inventor has devised an alternative solution to this problem. Rather than merely treating the inflammation of the local tissue, they have included acid scavenging agents in the stent. Hence the acidity of the breakdown products is neutralised upon degradation of polymers used in biodegradable stents. Thus the local inflammation is prevented rather than treated. It can be appreciated that this is a much more preferably approach to this problem. Until the present invention it had not been disclosed or suggested that such an approach could be used to addressing this problem.

Furthermore, the stent of the invention is prepared such that the acid scavenging agent is dissolved in the biodegradable material.

It is known in the art to prepare stents having pharmaceutically active agents coated on a surface, or incorporated in to the stent in voids, a matrix or as microparticles. In this way the stent can be used as a reservoir to deliver agents to a patient. However as can be appreciated this can lead to an unwanted unpredictability for the elution of the pharmaceutically active agent from the stent. This is especially the case for biodegradable stents since here eventually all of the stent is dissolved, which can mean there are pulses of release of the agent to the local environment. Moreover, the preparation of coated stents or stents including microparticles adds greatly to the cost of preparing the stents, since either specific coating techniques must be used which is costly due to the time involved and apparatus needed, or microparticles must be prepared which again is a lengthy process requiring specific apparatus.

The present inventor has devised an alternative solution to this problem. They have realised that it is possible to dissolve the acid scavenging agent in the biodegradable material used in the formulation for manufacturing of the stent. This can be readily achieved by heating a formulation of the biodegradable material and the acid scavenging agent to a temperature at which both the material and agent are melted. The agent then dissolves within the biodegradable material which results in a combined formulation in which the acid scavenging agent is fully and evenly dispersed. It can be appreciated that this approach provides a biodegradable stent in which the release characteristic of the agent is fully predicable and essentially stable over the time of its degradation and also the means of preparing the formulation is relatively quick. Moreover, existing means of preparing stents having pharmaceutically active agents often require the agent to be first dissolved in a solvent before it is coated or incorporated in to stent. By dissolving the acid scavenging agent in the biodegradable material this is obviated.

By “biodegradable stent” we include a generally tubular medical device which is implantable into a lumen in the human body. A stent is generally used to prevent, or counteract, a disease-induced, localized flow constriction in the lumen. The stent of the present invention is prepared preferably for use in a vascular lumen, for example a blood vessel. Preferably the stent is a coronary stent or a peripheral vascular stent. The stent may be self-expandable or balloon-expandable.

The stent is biodegradable. By biodegradable is meant that the material of the stent will undergo breakdown or decomposition into harmless compounds as part of a normal biological process.

Specific, types of biodegradable stent and their preparation are well known in the art.

The skilled person can readily prepare such a stent having the features of the stent of the invention using information available to them and provided herein. In particular, the method of the invention described below provides a biodegradable material having dissolved therein an acid scavenging agent. That material can then be used to prepare a range of different biodegradable stents.

For example, Su at al (2003) Annals Biomedical Engineering vol 31, 667-677 disclose a bioresorbable, expandable stent based on a linear, continuous coil array principle by which multiple furled lobes convert to a single lobe upon balloon expansion without heating. The document does not preparing the stent such that an acid scavenging agent is dissolved in the biodegradable material.

Grabow et al (2007) Annals Biomedical Engineering vol 35, 2031-2038 disclose a biodegradable balloon-expandable slotted tube stent. The document does not preparing the stent such that an acid scavenging agent is dissolved in the biodegradable material.

Colombo and Karvouni (2000) Circulation vol 102, 371-373 review a number of different biodegradable stent, including their structure and efficacy. The document does not preparing the stent such that an acid scavenging agent is dissolved in the biodegradable material.

The biodegradable stent of the invention is formed using a “biodegradable material”, i.e. a material which is broken down in situ following implantation into the body.

Biodegradable materials can be broadly classified into hydrolytically degradable polymers and enzymatically degradable polymers according to their mode of degradation.

Suitable biodegradable materials for the stent of the present invention include naturally derived or synthetic polymers as well as composites and combinations thereof and combinations of other biodegradable polymers. Biodegradable glass or bioactive glass is also a suitable biodegradable material for use in the present invention.

Representative examples of naturally derived polymers include albumin, collagen, hyaluronic acid and derivatives, sodium alginate and derivatives, chitosan and derivatives gelatin, starch, cellulose polymers (e.g., methylcellulose, hydroxypropyl cellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, and fibrinogen.

Synthetic biodegradable polymers and copolymers may be formed from one or more cyclic monomers (e.g. D-lactide, L-lactide, D,L-lactide, meso-lactide, glycolide, [epsilon]-caprolactone, trimethylene carbonate (TMC), p-dioxanone (e.g., 1,4-dioxane-2-one or 1,5-dioxepan-2-one), or a morpholinedione). In certain embodiments, the device include polymer fibers that comprise a plurality of glycolide and lactide (e.g., L-lactide, D-lactide, or mixtures thereof, also referred to as D,L-lactide) residues or meso-lactide). The ratio of glycolide to lactide residues in the copolymer may be varied depending on the desired properties of the fiber. For example, the polymer may have a molar ratio of glycolide residues that is greater than about 80; or greater than about 85; or greater than about 90; or greater than about 95. The fiber may be formed from a polymer having a 3:97 molar ratio of lactide (e.g., D,L-lactide) to glycolide, or a 5:95 molar ratio of lactide to glycolide, or a 10:90 molar ratio of lactide to glycolide.

Other suitable polymers include copolymers prepared from caprolactone and/or lactide and/or glycolide and/or polyethylene glycol (e.g., copolymers of [epsilon]-caprolactone and lactide and copolymers of glycolide and [epsilon]-caprolactone), poly(valerolactone), polydioxanone, and copolymers of lactide and 1,4-dioxane-2-one. Other examples of biodegradable materials include poly(hydroxybutyrate), poly(hydroxyvalerate), poly(hydroxybutyrate-co-hydroxyvalerate) copolymers, poly(alkylcarbonate), poly(orthoesters), tyrosine based polycarbonates and polyarylates, poly(ethylene terephthalate), poly(anhydrides), poly(ester-amides), polyphosphazenes, or poly(amino acids).

The following hydrolytically degradable polymers are particularly preferred in the preparation of the stent of the invention: polylactic acid including poly-L-lactic acid (PLLA) and poly-D,L-lactic acid (PDLLA), polyglycolic acid (PGA), and copolymers of polylactic acid, polyglycolic acid (PLGA); polycaprolactone, poly (4-hydroxybutyrate) (P4HB); polydioxanone; poly (trimethylene carbonate); poly (hydroxybutyrate-hydroxyvalerate); polyorthoester; poly(ester amides); poly (ortho esters); polyanhydrides; poly (anhydride-co-imide); poly (propylene fumarate); pseudo poly (amino acid); poly (alkyl cyanoacrylates); polyphosphazenes; polyphosphoester. Many of these materials are discussed in Nair et al (2007) Progress in Polymer Science 32, 762-798, including the structure of the polymers and how they can be sourced or prepared.

Biodegradable additives may be included in such polymers to aid their formation into stents; for example Poly(ethylene glycol) (PEG, MW 2000) can be used as a plasticizer to compromise the brittle mechanical properties of PLGA

The biodegradable stent can comprise more than one biodegradable material. For example, the stent may have backbone of one type of material, e.g. PLLA, coated with another biodegradable material, e.g. PDLLA; the stent may have a multilayered matrix, e.g. a PLLA/PLGA structure. The material can also be a blend of more than one polymer, e.g. PLLA and P4HB).

It is preferred that the biodegradable materials are PLGA or PLLA.

As mentioned above, PLGA is a L-lactide/glycolide copolymer. Various different ratios of L-lactide to glycolide monomer can be prepared as PLGA. Preferably the ratio is 85/15 L-lactide/glycolide. The preparation. of PLGA and PLLA is well known in the art and many routine laboratory protocols are known such that the skilled person could readily prepare PLGA or PLLA at different molecular weights without any inventive input. Moreover PLGA and PLLA biodegradable polymers materials can be obtained commercially from, for example, Purac (www.purac.com) as product reference Purasorb® PLG 8523.

The biodegradable stent of the invention is characterised as comprising an acid scavenging agent dissolved in the biodegradable material.

As discussed above, the hydrolysis of many biodegradable materials, including PLLA PDLLA, PGA, and PLGA copolymers of polylactic acid, polyglycolic acid, can produce acidic degradation products which can cause local inflammation at the site of the stent in the body. To alleviate the effect of the acidic degradation products, the present invention provides a stent including an acid scavenging agent. During the degradation of the stent in situ the acid scavenging agent neutralises the acidic degradation products and hence reduces the risk of local inflammation.

By “an acid scavenging agent” we include agents which can function in the body to neutralise the acidic degradation products.

Many compounds having this effect are known and can be used as such an agent, as will be appreciated by the skilled person. The following are examples of such agents. Pyrimido-pyrimidine compounds and its derivatives such as, for example, dipyridamole (2,6-bis(dithioethanolamino)-4,8-dipiperidinopyrimido(5,4-d)pyrimidine) and mopidamol (2,2′,2″,2′″-((4-(1-piperidinyl)pyrimido(5,4-d)pyrimidine-2,6-diyl)dinitrilo)tetrakisethanol), and derivatives or dipyridamole and mopidamol having the same pyrimido-pyrimidine structure. Pyrimido-pyrimidine compounds also include VK 744 and VK 774 as described in J Clin Pathol (1972) vol. 25, 427-432. Pyrimido-pyrimidine derivatives include pyrimido[5,4-d]pyrimidine, tetrachloro (2,4,6,8-tetrachloropyrimido[5,4-d]pyrimidine (available from Bepharm Ltd (www.bepharm.com)). Also RA25, which has the same substituents in all positions of the pyrimido ring of the nitrogens of the pyrimido pyrimidine ring. Further suitable agents include those pyrimido-pyrimidine compounds, and derivatives, disclosed in Schenone et al (2008) Current Drug Therapy vol. 3, 158-176; Walland, (1979) Pharmaceutisch Weekblad, 913-917; and U.S. Pat. No. 7,799,772.

Additional acid scavenging agents include coronary vasodilator or antiproliferative agents containing tertiary amino groups; bronchodilators containing amino groups, such as theophylline and its derivatives.

Dipyridamole (Persantine) and mopidamol are well known compounds readily available commercially or using standard synthesis techniques. Preferably the acid scavenging agent is dipyridamole and/or mopidamol.

By “dissolved therein” we mean that a formulation of the biodegradable material and the acid scavenging agent is heated until both components melt, such that the agent is homogeneously mixed and dissolved in the biodegradable material.

An example of methods that can be used to dissolve the acid scavenging agent in the biodegradable material is provided below and in the accompanying methods. It is preferred that the acid scavenging agent/biodegradable material formulation is prepared such that the acid scavenging agent is fully dissolved and evenly distributed throughout the biodegradable material.

According the first aspect of the invention provides a biodegradable stent comprising a biodegradable material having dissolved therein an acid scavenging agent, using the above mentioned biodegradable materials and acid scavenging agents.

The ratio between the biodegradable material and the acid scavenging agent can vary depending on the particular combinations used. For example where the biodegradable material is PLGA and the acid scavenging agent is dipyridamole, a ratio of 9 parts PLGA to 1 part dipyridamole can be used. A further example ratio is 8:2.

A preferred embodiment of the first aspect of the invention is where the acid scavenging agent is also a pharmaceutical agent.

An advantage of this embodiment, of the invention is that a single agent can be used to both provide the acid scavenging effect, and hence reducing local inflammation, and to also provide a further medicinal benefit to the patient following implantation into the body

The term “pharmaceutical agent” is well known in the art and includes chemical as well as natural substances having a benefit for the treatment or prevention of disease or disorder.

In particular, it is preferred that the pharmaceutical agent is an antiproliferative agent, coronary vasodilator agent and/or a bronchodilator.

By “antiproliferative agent” we include agents that inhibit cellular proliferation in the body. It is well known that there can be a proliferation of smooth muscle cells in response to the expansion of a foreign body against the vessel wall. Hence in the present embodiment the acid scavenging agent can also act to prevent such proliferation thus providing an advantage to the stent over those in the art.

For example, mopidamol and derivatives having the same pyrimido-pyrimidine structure has both an acid scavenging effect and has an antiproliferative effect. Hence a preferred embodiment of the first aspect of the invention is wherein the agent is mopidamol.

By “coronary vasodilator” we include agents that cause dilation of the coronary blood vessels, and hence alleviate the symptoms of reduced coronary blood flow associated with coronary artery disease.

For example, dipyridamole and derivatives having the same pyrimido-pyrimidine structure has both an acid scavenging effect and has a coronary vasodilator effect. Hence a preferred embodiment of the first aspect of the invention is wherein the agent is dipyridamole.

By “bronchodilator” we include agents that that dilates the bronchi and bronchioles, decreasing resistance in the respiratory airway and increasing airflow to the lungs. They are typically used to alleviate the symptoms of breathing difficulties, for example asthma and chronic obstructive pulmonary disease.

For example, theophylline and derivatives of that compound has both an acid scavenging effect and has bronchodilator effect. Hence a preferred embodiment of the first aspect of the invention is wherein the agent is theophylline.

A particularly preferred embodiment of the first aspect of the invention is where the stent comprises dipyridamole and mopidamol. Hence in this embodiment of the invention, the stent comprises agents providing an acid scavenging effect, antiproliferative effect and coronary vasodilator effect.

A preferred embodiment of the invention is wherein the stent further comprises one or more pharmaceutically active agents. Examples of such agents include the following classes of drugs: anti-proliferatives, such as growth factor antagonists, migration inhibitors, somatostatin analogues, ACE-inhibitors, and lipid-lowering drugs; anticoagulants, such as direct anti-coagulants which inhibit the clotting cascade, indirect anti-coagulants, which depress the synthesis of clotting factors, antiplatelet (aggregation) drugs, such as thromboxane A2 inhibitors or antagonists, adenosine inhibitors, glycoprotein receptor lib/IIIa antagonists, thrombin inhibitors; vasodilators, including vasoconstriction antagonists, such as ACE inhibitors, angiotensin II receptor antagonists, serotonin receptor antagonists, and thromboxane A2 synthetase inhibitors, and other vasodilators; anti-inflammatories; cytotoxic agents, such as anti-neoplastic agents, alkylating agents, anti-metabolites, mitotic inhibitors, and antibiotic antineoplastic agents; and radioactive agents or targets thereof, for local radiation therapy.

A further embodiment of the invention is wherein the stent comprises radio-opaque, echogenic materials and/or magnetic resonance imaging (MRI) responsive materials (i.e., MRI contrast agents) to aid in visualization of the device under ultrasound, fluoroscopy and/or MRI. For example, the stent may be made with or coated with a composition which is echogenic or radiopaque, e.g., made with echogenic or radiopaque with materials such as powdered tantalum, tungsten, barium carbonate, bismuth oxide, barium sulfate, metrazimide, iopamidol, iohexol, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface. Visualization of a device by ultrasonic imaging may be achieved using an echogenic coating. Echogenic coatings are well known in the art. For visualization under MRI, contrast agents (e.g., gadolinium (III) chelates or iron oxide compounds) may be incorporated into or onto the device, such as, for example, as a component in a coating or within the void volume of the device (e.g., within a lumen, reservoir, or within the structural material used to form the device), in some embodiments, a medical device may include radio-opaque or MRI visible markers (e.g., bands) that may be used to orient and guide the device during the implantation procedure. In another embodiment, these agents can be contained within the same coating layer as the compound or they may be contained in a coating layer (as described above) that is either applied before or after the layer containing the combination of compounds.

The present inventor has also developed an improved method for preparing a biodegradable material having dissolved therein an acid scavenging agent.

As discussed above, existing methods of preparing stents having pharmaceutically active agents typically involve coating agents on to surfaces of the stents, or embedding agents within voids or matrices, or encapsulating the agents in microparticles dispersed in the stent. However, a disadvantage of these approaches is that this can lead to an unwanted unpredictability for the elution of the pharmaceutically active agent from the stent, especially in the case of biodegradable stents.

To address this problem the inventor has devised a method of dissolving the acid scavenging agent within the biodegradable material.

Accordingly a second aspect of the invention provides a method of preparing a biodegradable material having dissolved therein an acid scavenging agent for use in the manufacture of a stent of the first aspect of the invention, comprising:
(i) preparing a formulation of the biodegradable material and the acid scavenging agent;
(ii) heating the formulation to melt the biodegradable material and the acid scavenging agent so as to dissolve the agent in the material; and
(iii) collecting and cooling the formulation of step (ii).

An example of such a process is provided in the accompanying example. Here it can be seen that the formulation of the biodegradable material (in that case PLGA) and the acid scavenging agent (in that case pyrimidine) was heated to around 180° C. to 190° C. At this temperature both the biodegradable material and the acid scavenging agent melted, providing a formulation with the acid scavenging agent dissolved therein. Following collection and cooling, the formulation can be prepared as fibres or strands which can subsequently be used in the preparation of stents according to the first aspect of the invention.

It is important to point out that the formulation of the biodegradable material and the acid scavenging agent must not be heated to a temperature at which the material or the agent starts to degrade since this could affect the performance of the stent prepared using the resulting formulation. Hence as can be appreciated this temperature will necessarily alter according to the specific biodegradable materials and the acid scavenging agents used. Since the melting temperature of each component will be known, then the skilled person can readily identify the correct temperature to which the formulation is to be heated so as to avoid any degradation.

A further advantage of this method of the invention over existing methods is that there is no need to use a solvent to dissolve the acid scavenging agent or any other pharmaceutically active agents. This is important since the use of solvents adds to the cost and time required for preparing a suitable formulation, and also care must be taken to ensure that the solvent can be safely used in the implantable stent. Furthermore most of the solvents are not suitable to be heated at the melting points of the polymers and the acid scavengers used in this invention.

An embodiment of the method of the invention is wherein the scavenging agent is also a pharmaceutical agent; preferably an antiproliferative agent, coronary vasodilator agent and/or a bronchodilator. Preferably the acid scavenging agent and antiproliferative agent is mopidamol or derivatives thereof. An alternative embodiment is where the acid scavenging agent and coronary vasodilator agent is dipyridamole or derivatives thereof. A further alternative embodiment is where the acid scavenging agent and bronchodilator is theophylline or derivatives thereof.

A further embodiment of the method of the invention is wherein the biodegradable material is PLLA or PLGA, the acid scavenging antiproliferative agent is mopidamol, and the ratio of PLLA or PLGA to mopidamol is 9:1 or 8:2. A still further embodiment is wherein the biodegradable material is PLLA or PLGA, the acid scavenging coronary vasodilator agent is dipyridamole, and the ratio of PLLA or PLGA to dipyridamole is 9:1 or 8:2.

A still further embodiment of the second aspect of the invention is where the biodegradable material is PLLA or PLGA and the acid scavenging agent comprises dipyridamole and mopidamol. Hence in this embodiment of the invention, the stent comprises agents providing an acid scavenging effect, antiproliferative effect and coronary vasodilator effect.

A third aspect of the invention provides a biodegradable material having dissolved therein an acid scavenging agent obtainable by the method of the second aspect of the invention.

A fourth aspect of the invention provides a method of preparing a biodegradable stent according to the first aspect of the invention comprising (i) performing the steps of the method of the second aspect of the invention; and (ii) preparing a stent comprising the prepared formulation.

A fifth aspect of the invention provides a stent substantially as described herein by reference to the description and figures.

The invention will now be further described with reference to the following examples and Figures.

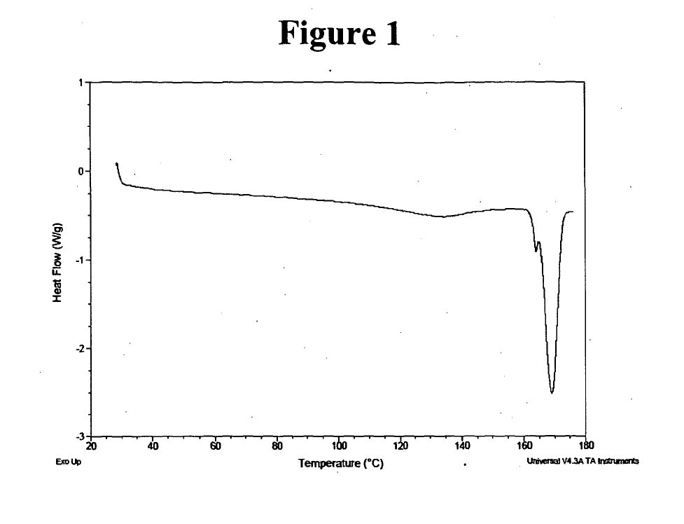

FIG. 1: Thermogram of dipyridamole

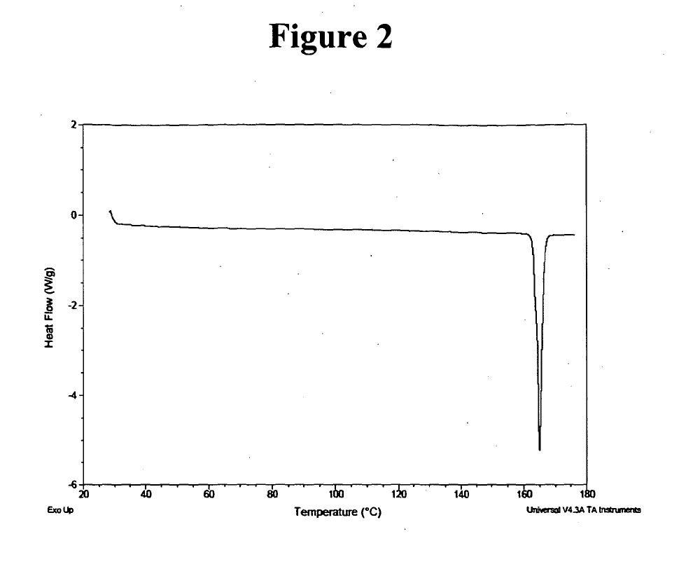

FIG. 2: Thermogram during reheating dipyridamole cooled after melting in the DSC sample holder.

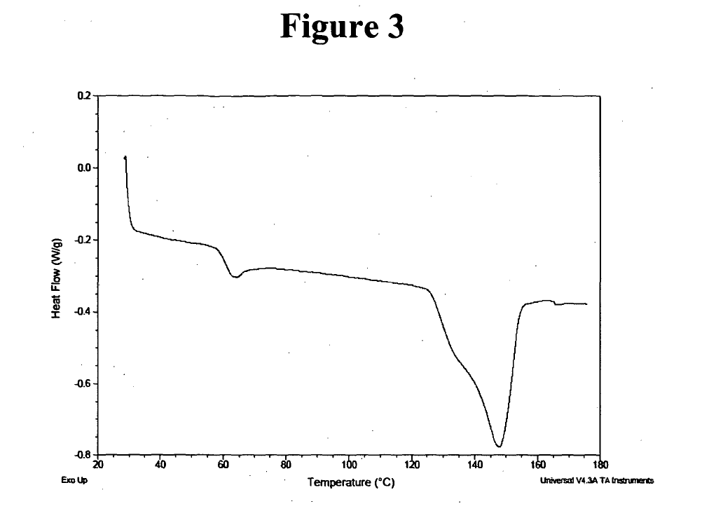

FIG. 3: Thermogram of L-Lactide/Glycolide copolymer (Purasorb® PLG 8523)

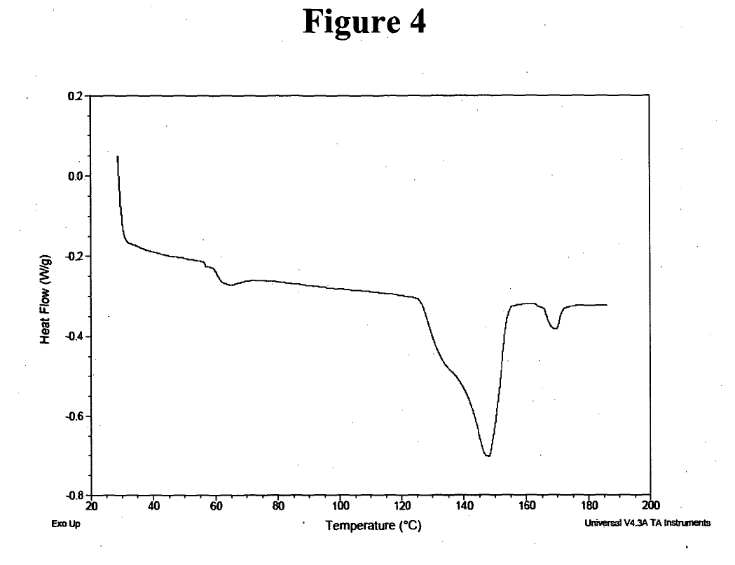

FIG. 4: Thermogram of physical mixture of dipyridamole and Purasorb® PLG 8523.

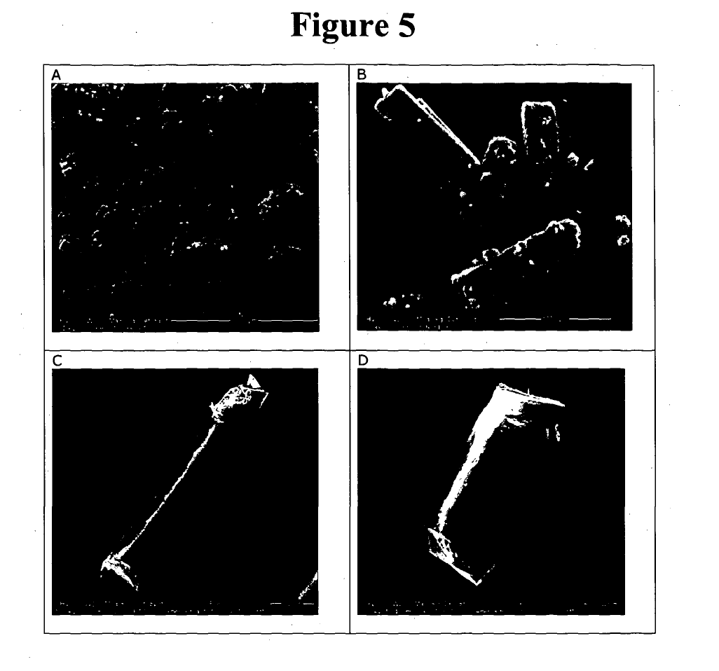

FIG. 5: SEM photomicrographs of dipyredimole (A and B) and drug loaded strands extruded at 170° C. (C) and 140° C. (D).

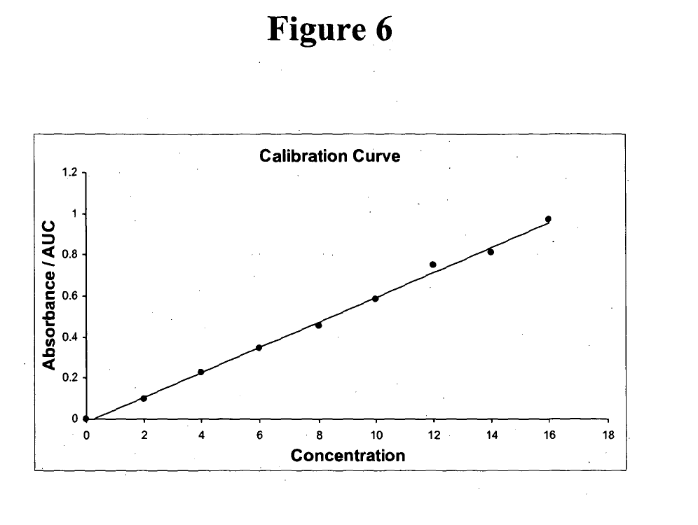

FIG. 6: Calibration curve for dipyridamole in Phosphate buffer pH 7.4.

### Example 1

### Drug Formulations for Cardiovascular Stents

### Background

Cardiovascular disease (CVD) is a major health concern facing the UK and indeed the rest of the world. Coronary stents are permanently implantable devices that are placed percutaneously in coronary arteries to recover and maintain normal blood flow. Metallic coronary stents were one of the most important advances in the treatment of CVD during the last two decades but restenosis (re-narrowing) and late thrombosis are major associated problems.

Historically and to the present day the coronary stent market has been based upon the development of bare metallic and drug-coated metallic coronary stents. Following implantation bare metallic stents result in remodelling the interior walls of coronary arteries. Once this process is complete these stents cease to be a positive factor in cardiovascular therapy and can have negative consequences in the long term, leading to tissue necrosis and “stent jailing,” where access to subsidiary vessels through the stent becomes difficult or impossible. Additionally, drug coated stents have sometimes led to poor endothelialisation (vascular cell covering) of the stent which, when it loses its coating, comes into direct contact with blood, and thereby triggers a coagulation cascade and leads to stent thrombosis.

The inventors have devised a range of biodegradable drug eluting coronary stents. The unique properties of these stents are that once they have caused vessel remodelling and have been encapsulated in the vessel wall, they biodegrade after a period of 12 to 18 months. The development of fully biodegradable stents that perform the necessary function of remodelling the arterial flow area and then disappear, could make a radical contribution to the priority of tackling a disease that is one of the greatest contributors to mortality in the UK and worldwide.

Although some clinical trials have already taken place for the first generation of fully biodegradable stents, the opportunity still exists to gain a foothold in the US dominated, multi-billion dollar stent market. In a recent expert review, fully biodegradable coronary stents were stated to hold the greatest promise for tackling the challenge of designing a safe drug-eluting coronary stent.

### The present Invention

There are two distinct areas of focus that will set the present invention of the biodegradable coronary stents formulations apart from other stents:

(1) Blood Compatibility, Anti-Proliferative and Acid Scavenger Agents

One of the main concerns about the existing clinically-approved biodegradable polymers (based on PLLA and PLGA polymers) is the formation of acidic molecules during the water hydrolysis process, which occurs in the body causing inflammation in the artery. In the present invention, it has been identified blood-compatible agents with a long history of clinical use such as Dipyridamole (Persantine) and derivatives, which can also act as an acid scavenger in the artery. Each molecule can be used for these dual actions once it has been formulated in the stent construction. Another agent with dual actions as anti-proliferative and an acid scavenger such as mopidamol and derivatives has been also identified. Those agents would be able to produce a long-term biocompatible and non-inflammatory biodegradable stents

(2) Drug Delivery

Most stents today have the drug of choice spread throughout the coating or filling small wells on the surface of the device, so that drug is delivered throughout the entire surface of the device. With the new family of stent designs, it is the intention that the drug will be focused in a specific area to target the most significant area of disease. This area will usually be within the mid-point of the stent. Furthermore the area of drug delivery will be marked with an x-ray visible marker, so that the stent can be accurately positioned with regard to the specific location of the disease in the vessel.

### Example 2

### A Formulation of a Biodegradable Material Having Incorporated Therein an Acid Scavenging Agent

Introduction:

Experiments were performed to investigate polymer and drug combinations suitable for the production of a fully biodegradable drug loaded stent. Formulations of polymer and drug were produced using a pharmaceutical grade twin screw extruder and the flow behaviour and drug release kinetics of the compounds was be quantified. Measurement of the thermal properties and flow behaviour of the formulations informs the process of designing a suitable manufacturing route (e.g. moulding, extrusion, woven fibre).

Blends of Biodegradable Materials and Acid Scavenging Agents

Selection of polymer and Active Pharmaceutical Ingredient (API)

Polymer: L-lactide/glycolide copolymer (Purasorb PLG 8523).
Solubility: methlyene chloride, chloroform, hexafluoro-isopropanol
Tg: 55-60° C.
Melting point: 140-150° C.

API: Dipyridamole (persantine)
Melting point: 163° C.
Molecular Weight: 504.317
Water solubility: slightly soluble in water
Soluble: Ethanol, dimethyl sulfoxide

Methods:

1. Differential Scanning Calorimetry (Thermal Characterization)

Thermal profiles were generated in the range of 25 to either 180 or 190° C. using a TA instruments Q2000 DSC with RCS90 cooling unit. Temperature calibration was performed using an indium metal standard supplied with the instrument at the respective heating rate. Accurately weighed samples (1.5-2.5 mg) were placed in aluminium pans using similar empty pans as a reference. A heating rate of 10° C. min-1 was employed and an inert atmosphere was maintained by purging nitrogen gas at a flow rate of 50 ml/min.

2. Extrusion of Dipyridamole Loaded PLGA Strands

Dipyridamole and PLG in 1:9 weight ratios were blended and extrusion was carried out using a co-rotating twin screw extruder (Minilab, Thermo Scientific, UK). Powdered material was fed into the extruder maintained at three different temperatures and run at 40 rpm screw speed. The resultant strands were collected, allowed to cool and stored.

3. Scanning Electron Microscopy:

Samples were mounted on aluminium pin-stubs (Agar Scientific, Stansted, U.K.) for SEM using self adhesive carbon mounts (Agar Scientific). The mounted samples were examined using an FEI Quanta 400 Scanning Electron Microscope (Cambridge, U.K.) in high vacuum operated at an acceleration voltage of 20 kV. XTM Microscope control software version 2.3 was used for imaging.

4. Dissolution Study:

Medium: Phosphate Buffer Saline pH 7.4

Dissolve 2.38 g of disodium hydrogen orthophosphate, 0.19 g of potassium dihydrogen phosphate and 8.0 g of sodium chloride in sufficient water to produce 1000 ml and adjust the pH if necessary.

Dissolution Method:

The extruded strands were cut into sufficient length equivalent to 300 μg drug load. The strands were dropped in 15 ml phosphate buffer saline pH 7.4 in a beaker, maintained at 37° C. and stirred at 50 rpm. The samples were withdrawed after 1, 2, 5, 7, 14, 21, 28 days and analyse for dipyridamole concentration using UV spectrophotometer at 294 nm, The dissolution study was carried out on two trails conducted at 170 and 140° C. in triplicate and data is reported as average and standard deviation.

Results and Discussion:

1. Differential Scanning Calorimetry (Thermal Characterization)

Thermogram of dipyridamole showed a melting endotherm at 168° C. (FIG. 1). This was slightly higher than the reported melting point in the literature (163-167° C.). However, when the same sample holder was cooled and reheated, a sharp melting endotherm was observed at 165° C. (FIG. 2). This indicates that the drug do not form glass on cooling the melt. It should be noted that the cooling rate was not fast enough to effect melt quenching. The cooling after extrusion is also not too fast and similar observation can be expected during extrusion. Thermogram of L-Lactide/Glycolide copolymer (Purasorb® PLG 8523) showed first endotherm at 60-65° C., which attributed to Tg of polymer and second at 148° C. attributed to the melting of polymer (FIG. 3). These results are in accordance with the reported values (Tg=50-60° C. and M.P at 140-150° C.) for the polymer.

Thermogram of the physical mixture of the drug and polymer showed first endotherm at 60-65° C. attributed to Tg and second at 148° C. attributed to the melting of polymer, followed by third endotherm at 168° C. attributed to the melting of the drug (FIG. 4). As the melting endotherm corresponding to drug was small it can be inferred that part of the drug did not dissolve in the molten polymer and this remaining drug melts only after reaching 168° C. (its melting point). Therefore, if the processing temperature is kept below melting point of the drug, it may result in the dispersion of drug partly in the form of crystals and partly in the amorphous form.

Therefore, the processing temperature will dictate whether formulation will have amorphous or crystalline form of the drug. However, the thermal studies are carried out in the absence of shear and during extrusion variable shear is applied. This may influence the dispersibility of drug in polymer and its state.

2. Extrusion of Drug Loaded Polymer Strands:

The particle size of the polymer is too big compared to the drug. Due to this difference in the particle size uniform blending of drug and polymer could not be achieved. Therefore, an attempt was made to grind the polymer pellets down to the size comparable to the particle size of drug. However, the polymer was difficult to mill and caused local temperature to rise resulting in excessive load on mill, leading to its tripping. Therefore, we made an attempt to use liquid nitrogen purging in order to maintain the local temperature. However, we could not succeed in milling polymer to the desirable size. Finally the polymer was blended as received with the drug in the weight ratio of 1:9 for extrusion.

Considering the results of thermal analysis, two batches were extruded, one above the melting temperature of drug (at 170° C.) and other below the melting temperature (at 140° C.). The batch processed at 170° C. resulted in a low viscosity strand which was difficult to handle, though it was clear transparent yellow strand. This indicates the drug might have been dissolved completely in the polymer melt giving and clear and uniform dispersion of the drug. The batch processed at 140° C. resulted in an opaque yellow strand with high viscosity and manageable strength. This might be due to incomplete dissolution of the drug in the polymer melt. Both the strands were collected and stored in a desiccators before analysis.

3. Scanning Electron Microscopy:

Dipyridamole showed mixture of rod shaped cubic crystals of variable size ranging from 20 to 200 μm (FIG. 5A and B). The extruded stands showed rods of around 1 mm diameter with smooth surface. There was no much difference in the appearance of stands extruded at 140 and 170° C. as observed in SEM images (FIG. 5C and D, respectively).

4. Dissolution

The calibration curve was constructed in the phosphate buffer saline pH 7.4 in the range of 2 to 16 pg. The curve was linear with the R2=0.9986. The slope was 13.39 and constant=0.265. The curve is presented in FIG. 6.

The dissolution study was carried out on the strands equivalent to 300 pg of drug loading. The dissolution study was planned for 28 days and the study is complete until 14 days. The strand was intact with no signs of erosion. The release was found to be in non detectable level and hence marked as zero percent till 14 days. In summary, the rate of drug release depends on the time of deg ration of the polymer used.

Conclusion

The polymer and drug suitable for developing and manufacturing the drug eluting stents are selected. The processing conditions suitable for achieving drug and polymer miscibility are also optimized. The feasibility of extruding these formulations was studied and no degradation was observed during extrusion. Thermal analysis also indicates no degradation. Polymer erosion and drug release properties are acceptable.

1. A biodegradable stent comprising a biodegradable material having dissolved therein an acid scavenging agent.

2. The stent of claim 1 wherein the biodegradable material is a hydrolytically degradable polymer.

3. The stent of claim 2 wherein the polymer is selected from the group consisting of: polylactic acid (PLA, PLLA, PDLLA), polyglycolic acid (PGA), a copolymer of polylactic acid and a copolymer of polyglycolic acid (PLGA).

4. The stent of claim 1 wherein the stent comprises more than one biodegradable material.

5. The stent of claim 1 wherein the acid scavenging agent is selected from the group consisting of: dipyridamole, mopidamol, a derivative of dipyridamole having a pyrimido-pyrimidine structure;
a derivative of mopidamol having a pyrimido-pyrimidine structure, theophylline and derivatives of theophylline.

6. The stent of claim 5 wherein the acid scavenging agent is selected from the group consisting of: dipyridamole, mopidamol and a combination of dipyrimadole and mopidamol.

7. The stent of claim 1 wherein the acid scavenging agent is also a pharmaceutical agent.

8. The stent of claim 7 wherein the pharmaceutical agent is selected from the group consisting of an antiproliferative agent, a coronary vasodilator agent and a bronchodilator.

9. The stent of claim 8 wherein the antiproliferative agent is selected from the group consisting of: mopidamol and derivatives thereof.

10. The stent of claim 8 wherein the coronary vasodilator agent is selected from the group consisting of: dipyridamole and a derivative thereof.

11. The stent of claim 8 wherein the bronchodilator is selected from the group consisting of: theophylline and a derivative thereof.

12. The stent of claim 9 wherein the biodegradable material is selected from the group consisting of: PLLA and PLGA, the acid scavenging antiproliferative agent is mopidamol, and the ratio of biodegradable material to mopidamol is 9:1 or 8:2.

13. The stent of claim 10 wherein the biodegradable material is selected from the group consisting of: PLLA and PLGA, the acid scavenging coronary vasodilator agent is dipyridamole, and the ratio of biodegradable material to dipyridamole is 9:1 or 8:2.

14. The stent of claim 1 wherein the stent comprises dipyridamole and mopidamol.

15. The stent of claim 1 comprising a further pharmaceutically active agent.

16. The stent of claim 1 in the form of a generally tubular body.

17. The stent of claim 1 further comprising radio-opaque, echogenic material and/or magnetic resonance imaging (MRI) responsive material.

18. A method of preparing a biodegradable material having dissolved therein an acid scavenging agent for use in the manufacture of claim 1, comprising:
(i) preparing a formulation of the biodegradable material and the acid scavenging agent;
(ii) heating the formulation to melt the biodegradable material and the acid scavenging agent so as to dissolve the agent in the material; and
(iii) collecting and cooling the formulation of step (ii).

19. The method of claim 18 wherein the formulation is heated to above the melting point of the biodegradable material and the acid scavenging agent.

20. The method of claim 18 wherein the method does not use a solvent to solubilise the biodegradable material or the acid scavenging agent.

21. The method of claim 18 wherein the acid scavenging agent is also a pharmaceutical agent.

22. The method of claim 18 wherein the biodegradable material is selected from the group consisting of PLLA and PLGA, and the acid scavenging agent is mopidamol, and the ratio of biodegradable material to mopidamol is 9:1 or 8:2.

23. The method of claim 18 wherein the biodegradable material is selected from the group consisting of PLLA and PLGA, and the acid scavenging agent is selected from the group consisting of dipyridamole and derivatives thereof

24. The method of claim 18 wherein the biodegradable material is selected from the group consisting of PLLA and PLGA and the acid scavenging agent comprises dipyridamole and mopidamol.

25. A biodegradable material having dissolved therein an acid scavenging agent obtainable by the method of claim 18.

26. A method of preparing a biodegradable stent comprising a biodegradable material having dissolved therein an acid scavenging agent, comprising (i) performing the steps of the method of claim 18; and (ii) preparing a stent comprising the prepared formulation.

27. (canceled)

28. The method of claim 21 wherein the pharmaceutical agent is selected from the group consisting of an antiproliferative agent, a coronary vasodilator agent and a bronchodilator.
